(19) United States
(12) Patent Application Publication
(10) Pub. No.: US 2002/0071570 A1
(43) Pub. Date: Jun. 13, 2002

(54) Hybrid structure

(76) Inventors: Gerard Cohen, Rosh Ha' ayin (IL); Alexander Strovinsky, Rishon Lezion (IL)

Correspondence Address: BROWDY AND NEIMARK, P.L.L.C., 624 NINTH STREET, NW, SUITE 300, WASHINGTON, DC, 20001-5303, US

(21) Appl. No.: 10/002,173

(22) Filed: Dec. 5, 2001

(30) Foreign Application Priority Data
Dec. 7, 2000 (IL) 140148

(51) Int. Cl. A61B 7/04

(52) U.S. Cl. 381/67

(57) ABSTRACT

A hybrid stethoscope which enables a physician who places the bell of the instrument on a body site in order to hear sounds emanating from an underlying internal region, to concurrently see an analog waveform of these sounds. The combined visual and aural impressions produced thereby facilitate an analysis of the sounds and a diagnosis of the disorder giving rise thereto. Attached to the bell so that it is viewable by the physician handling the bell is a self-sufficient visual display module provided with a transducer that picks up sounds received by the bell to yield an audio signal that is fed to an LCD display on whose screen is exhibited the analog waveform of the sounds. Thus a physician hears sounds emanating from an internal body region within the ambit of the bell and at the same time effectively sees these sounds and thereby gains greater insight into the is character of the sounds and their medical significance.

### FIELD OF THE INVENTION

This invention relates generally to stethoscopes and more particularly to hybrid stethoscopes adapted to convey to a physician's ears sounds emanating from an internal body region and at the same time to convey to the physician's eyes a visualization of these sounds.

### STATUS OF PRIOR ART AND BACKGROUND OF THE INVENTION

Ausculation refers to the act of listening to sounds originating in internal organs of the body as an aid to diagnosing disorders of these organs and to the prescription of appropriate treatments therefor. The instrument commonly used for ausculation is the acoustic stethoscope. This consists of a bell placed by the physician on a body site overlying the organ of interest, the sounds received by the bell being conveyed by a tubular line to acoustic earphones inserted in the physician's ears.

Because a stethoscope is a non-invasive means for ausculation, it is an invaluable tool for the medical diagnosis of abnormalities, particularly cardiac, lung and vascular disorders. However, the interpretation of sounds originating in internal organs of the body to determine their medical significance is not a routine matter. It takes a fair degree of skill on the part of a physician to make sense of these sounds. One reason these sounds are difficult to interpret is because of limitations of a stethoscope.

A conventional acoustic stethoscope functions as a low-pass filter whose band pass characteristics vary to a substantial degree from manufacturer to manufacturer. But in all cases, high-frequency components of the internal body sounds are filtered out and therefore make it more difficult to determine the medical significance of the sounds.

To some degree, the limitations of an acoustic stethoscope are overcome in an electronic stethoscope in which the body sounds are sensed by a microphone to produce an audio signal that is amplified and then applied to electromagnetic earphones. But an electronic stethoscope, because it is highly sensitive, also picks up internally-generated random noises. These mask the more meaningful sounds and confuse the listener.

Also available are acoustic stethoscopes having a double-headed bell, one head being adapted to engage a smooth body surface, the opposite head, a hairy body surface. In all other respects, this stethoscope behaves like a conventional stethoscope and has the same acoustic limitations.

Another form of acoustic stethoscope is the stereophonic stethoscope or “symballophone” which makes it possible for the ears of the physician to compare the intensity and quality of sounds arising from the internal organs, thereby informing the physician of the location of the source of the sounds. But this acoustic stereophonic stethoscope also acts as a low-pass filter which excludes high-frequency components of the sounds.

To facilitate an in-depth analysis of sounds picked up by an acoustic stethoscope, there is disclosed in Bredesen et al. U.S. Pat. No. 5,213,108 a stethoscope having attached to its bell a transducer that intercepts the body sounds received by the bell to yield an audio signal. The signal is conveyed by a cable to an external monitor. This monitor is provided with a liquid crystal display which presents on a screen an analog waveform of the audio signal derived from the internal body sounds.

In this monitor, the incoming audio signal is processed via a sampled analog-to-digital waveform whereby the sounds represented by the waveform can be manipulated to carry out timing and other measurements for assisting a physician in his analysis of the body sounds and in diagnosing the disorders responsible therefore.

The entire disclosure of the Bredesen et al. patent is incorporated herein by reference, for associated with a hybrid stethoscope in accordance with the invention is an external monitor which executes similar functions to aid in the analysis of sounds originating in an internal body region.

Absent in Bredesen is the key feature of the present invention which is a visual display module mounted on fie stethoscope bell which displays to the physician handling the bell a visual representation of the body sounds being received by the bell. As a consequence, the physician effectively sees as well as hears the internal body sounds, the impressions made thereby being concurrent, thereby affording the physician with greater insight into the character of the sounds and their significance.

The reason visualization of the internal body sounds affords greater insight as to the nature of these sounds and their medical significance is that visualization overcomes not only the acoustic limitations of an acoustic stethoscope bit also the inherent limitations of the human ear.

Audible sounds lie in a frequency range of about 10 Hz to 15500 Hz. But few ears are capable of hearing the full audio ranges and with age or as a result of ear disorders, many ears become markedly less sensitive to high-frequency components in this range.

Hence in the context of body sounds heard via a stethoscope, many physicians because of ear imperfections gain a distorted or an inadequate impression of the character of sounds originating in an internal body region. As a consequence, the physician may misinterpret the medical significance of these sounds.

### SUMMARY OF THE INVENTION

In view of the foregoing, the main object of this invention is to provide a hybrid stethoscope which enables a medical practitioner who manually places the bell of the stethoscope on a body site to hear sounds emanating from an internal region underlying the site and to concurrently visualize these sounds.

More particularly, an object of the invention is to provide a hybrid stethoscope having attached to its bell a self-sufficient visual display module which exhibits an analog waveform of sounds received by the bell. Hence a physician handling the bell and hearing these sounds also effectively sees the sounds, the combined visual and aural impressions enhancing the physicians' understanding of the medical significance of the sounds.

A major advantage of a hybrid stethoscope is that its visual display is presented on the bell receiving the sounds being analyzed, the combined sensory impressions facilitating an analysis of the sounds and a diagnosis of the disorder indicated thereby. Another advantage of the invention is that the visual display module is attachable to a standard, commercially-available acoustic stethoscope, thereby converting tie stethoscope into a hybrid stethoscope at a relatively low cost.

The role of visualization in enhancing the comprehension of a listener to sound can be demonstrated by lip-reading experiences. A listener who has difficulty in understanding the meaning of sounds issuing from the mouth of a speaker, by observing the changing configurations of the speaker's lips as the sounds are being produced, can gain a clearer impression of the nature of the sounds.

By the same token, viewing the varying shape and amplitude of a waveform representing the sounds being received by the bell of the stethoscope imparts to the listener to these sounds a clearer impression of their nature, especially when the waveform is displayed adjacent the source of the sounds, thereby coordinating the visual and aural impressions.

Still another advantage of a hybrid stethoscope in accordance with the invention is that it makes possible better screening of a cardiac condition thereby making it unnecessary to refer the patient to a specialist for further examination.

The module included in the hybrid stethoscope provides a visual display of the waveform of the sounds being picked up by the stethoscope and when the physician is presented with a marked disparity therebetween he then recognizes the need to clarify the condition of the patient by means of an echo doppler examination.

Also an object of this invention is to provide an external monitor linked by a microwave or infrared carrier to the hybrid stethoscope, the monitor acting to manipulate the waveform of the sounds received by the bell of the stethoscope to effect measurements of timing and other parameters which assist in the diagnosis of the abnormalities indicated by the internal body sounds.

Briefly stated, these objects are accomplished by a hybrid stethoscope which enables a physician when placing the bell of the instrument on a body site in order to hear sounds emanating from an underlying internal region, to concurrently see an analog waveform of these sounds. The combined visual and aural impressions produced thereby facilitate an analysis of the sounds and a diagnosis of the disorder giving rise thereto.

Attached to the bell so that it is viewable by the physician handling the bell is a self-sufficient visual display module provided with a transducer that picks up sounds received by the bell to yield an audio signal that is fed to an LCD display whose screen exhibits an analog waveform of the sounds. Thus a physician using the stethoscope hears sounds emanating from an internal body region within the ambit of the bell, while at the same time effectively sees these sounds and thereby gains greater insight into the character of the sounds and their medical significance.

### BRIEF DESCRIPTION OF THE DRAWINGS

For a better understanding of the invention, as well as other objects and features thereon reference is made to the annexed drawings wherein:

FIG. 1 shows a hybrid stethoscope in accordance with the invention;

FIG. 2 is a separate view of the visual display module attached to the stethoscope;

FIG. 3 illustrates the display module attached to a double-headed stethoscope bell;

FIG. 4 shows the external monitor linked to the hybrid stethoscope;

FIG. 5 schematically illustrates the components of a module provided with a microwave transmitter to convey the data developed by the module to an external monitor; and

FIG. 6 is a block diagram of diverse external sites to which the module is capable of transmitting data; and

FIG. 7 illustrates the position of the stethoscope on the body of a patient in different modes of operation.

### DETAILED DESCRIPTION OF THE INVENTION

The Hybrid Stethoscope:

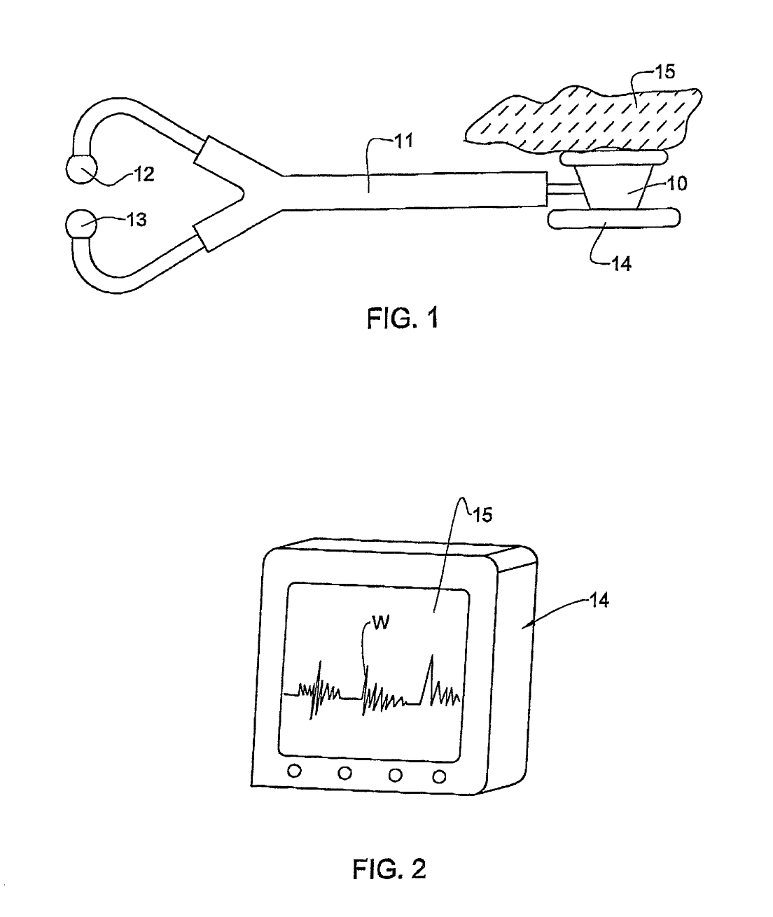

Shown in FIG. 1 is a hybrid stethoscope in accordance with the invention, the instrument including standard acoustic bell 10 having a diaphragm. In use, bell 10 is placed by a medical practitioner on a body site B overlying an internal region thereof such as the heart region, so as to pick up sounds emanating therefrom.

Bell 10 is coupled by a tubular line 11 acting as an acoustic wave guide to a pair of acoustic earphones 12 and 13 which are inserted in the ears of the physician using the stethoscope. The physician can therefore hear sounds emanating from the internal body region within the ambit of the bell.

Attached to the rear end of bell 10 is a self-sufficient battery-powered visual display module 14. Module 14 is provided with a liquid crystal display (LCD) device 15 or a similar miniature device having the same function on whose screen is exhibited an analog waveform W of the audible sounds impinging on the diaphragm of the bell and conveyed to the ears of the physician.

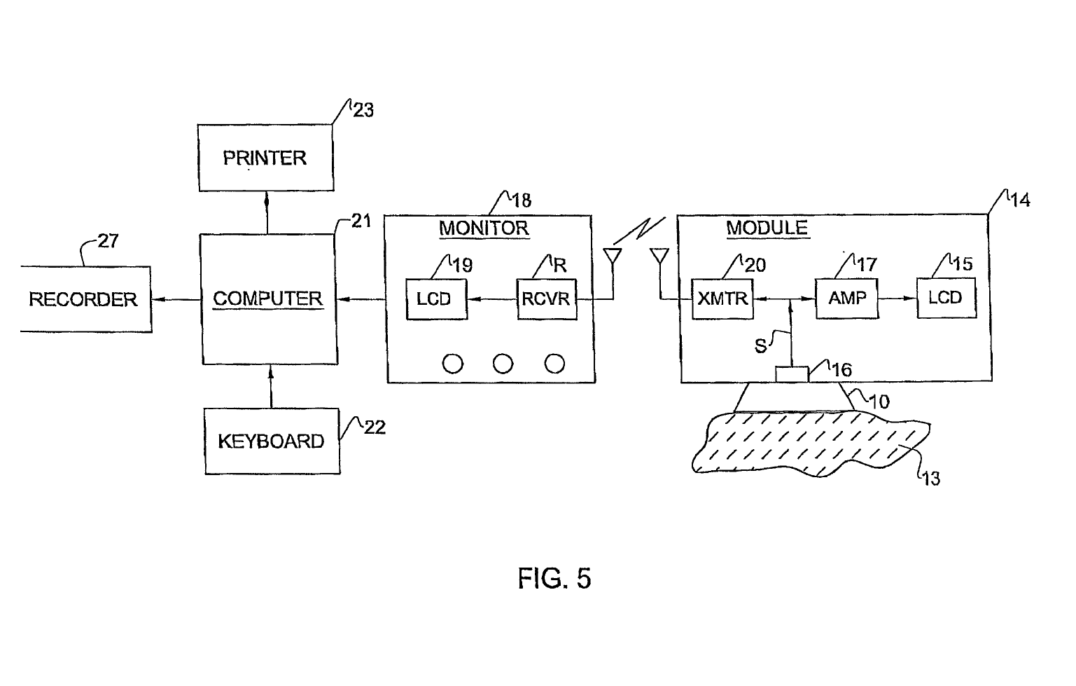

As shown schematically in FIG. 5, module 14 is provided with a microphone 16 that is placed to intercept sounds emanating from the internal body site region and received by bell 10. In practice, the bell may be provided at its rear end center with an orifice in registration with the microphone of the module.

Microphone 16 yields an audio signal S that represents substantially the full frequency range of the internal body sounds. However, as previously noted, the acoustic stethoscope acts as a low-pass filter, hence what is heard by the physician in whose ears are inserted the earphones 12 and 13, are only those frequency components of the sounds which pass trough the stethoscope, not the fall frequency range.

Signal S from microphone 16 is applied to an integrated-circuit amplifier 17 whose output is fed to liquid crystal display device 15 on whose screen is exhibited the waveform W of the audio signal derived from the internal body sounds.

By way of example, we shall consider the use of the hybrid stethoscope by a physician to listen to sounds emanating from the heart region of a patient. The character of these sounds depends on the cardiac disorder responsible therefor. Thus heart murmurs are atypical sounds indicative of functional or structural abnormalities. Different sounds are generated as a result of disturbances in the natural rhythm of the heart a condition known as arrhythmia. A defective heart valve also produces distinctive sounds.

In order for a physician to make sense of sounds emanating from the region of the heart, he must, as it were, have a full picture of these sounds. This full picture is provided by the hybrid stethoscope which makes it possible for its user, as he listens to sounds emanating from an internal body region, to effectively see these sounds, and from the combined visual and aural impressions to obtain a full picture thereof.

A hybrid stethoscope in accordance with the invention is compatible with existing medical procedures. In a typical medical facility in which patients are distributed throughout the rooms of a hospital, a physician doing the rounds and going from patient to patient invariably cares on his person an acoustic stethoscope, this usually being the only tool, as it were, carried by the doctor.

Because the hybrid stethoscope only differs from a standard stethoscope by a small visual display module attached to the bell, this addition does not alter the portability of the instrument which is just as easy to carry and to use as a standard stethoscope.

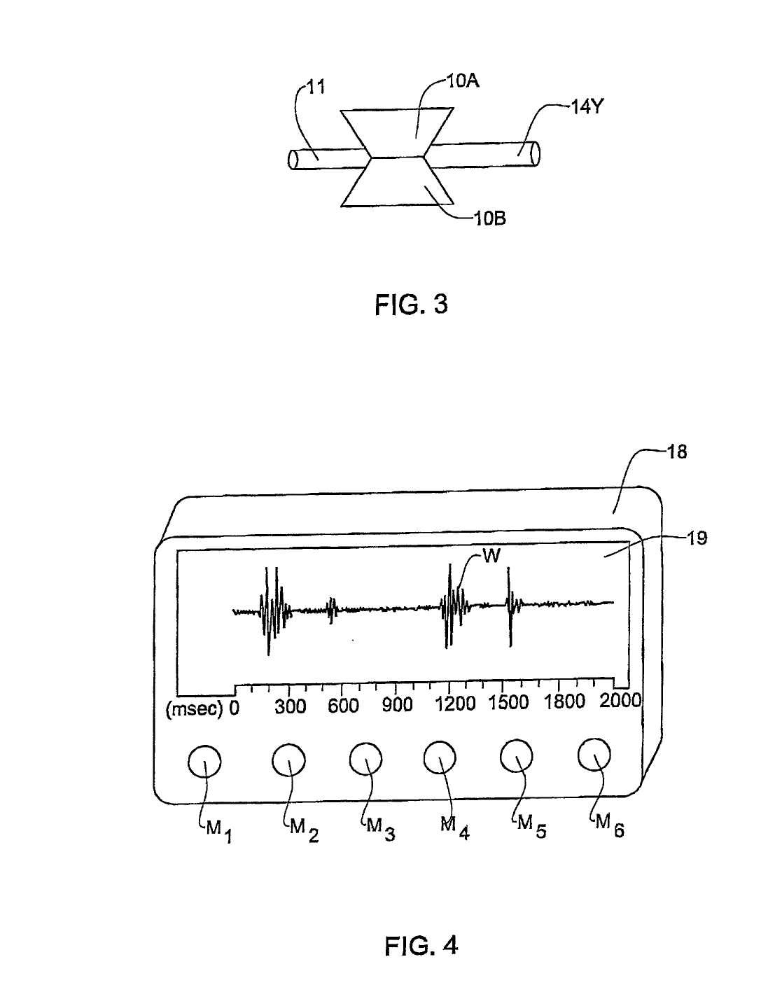

Some acoustic stethoscopes have a double-headed bell, as shown in FIG. 3, one head 10A being adapted to engage a smooth body skin, the opposite head 10B a hairy body skin whose hairs interfere with the operation of a conventional bell. In his double-head bell, the visual display module 14X is mounted laterally from the junction between the rear ends of the abutting bells.

To create a wireless link between the module and an external monitor 18 included in module 14 is a miniature, integrated-circuit microwave transmitter 20 whose radio carrier is modulated by audio signal S yielded by microphone 16. The transmission from the module on the bell of the stethoscope is intercepted by a receiver R incorporated in external monitor 18. The resultant audio output of receiver R is fed to LCD display 19 of the monitor on whose screen is presented along a millisecond scale the waveform of the internal body sounds. Monitor 18 includes menu keys M1 to M6 which can be actuated to select the desired function or measurement to be made.

In practice, a single monitor may be used to service several hybrid stethoscopes. To this end, the transmitter on each stethoscope which sends out a microwave data signal precedes tis data with a bar code signal identifying the stethoscope. Hence when a microwave transmission from a particular stethoscope is received by the monitor and processed therein, the resultant data whose source is identified, can be stored in the database of the monitor.

The External Monitor:

A portable hybrid stethoscope of the type shown in FIG. 1 enables a physician to visualize as well as to hear internal body sounds and from the combined aural and visual impression to determine the nature of the disorder responsible for the sounds.

However, should one wash to make an in-depth analysis of waveform W, displayed in module 14 attached to the bell of the stethoscope, to more accurately determine the medical significance of the sounds, it is then necessary, in effect, to dissect or manipulate this waveform in order to measure timing and other parameters that are related to particular disorders.

Useful for this purpose is a monitor similar to that shown in the above-identified Bredesen patent which is adapted to manipulate the waveform data, such manipulation including real time analog filtering of the displayed waveform, digital filtering of the stored waveform and internal timing between strategic positions of the body sound waveforms. The Bredesen monitor aids a physician in the analysis, recognition and diagnosis of the abnormalities responsible for the internal body sounds.

The box-like monitor of Bredesen is coupled by a cable to a microphone attached to the bell of an acoustic stethoscope and the resultant combination is not a portable instrument that can be carried conveniently by a physician. Moreover, the Bredesen arrangement though it presents the waveform of the internal body sounds being heard by the physician, it does not present this waveform where it can easily be seen by the physician as he holds the bell of the stethoscope. The monitor of Bredesen is necessarily placed at some distance from the bell of the stethoscope, and a physician cannot as a practical matter, both be looking at the patient as he manipulates the bell and be looking at the monitor. But even if he could then see the monitor, the waveform presented on its screen would be too far away from the patient to be clearly discernable by the doctor,

In the hybrid stethoscope, the visual display of the waveform appears on the rear of the bell receiving the body sounds. Hence the physician holding the bell and listening to the internal body sounds is able to simultaneously observe the waveform. Though the waveform is on a small scale, it is not far from the eyes of the physician and therefore is easily observed.

In the present arrangement, instead of a cable connection between a monitor and a stethoscope as in the Bredesen patient, there is a wireless link between the visual display module attached to the stethoscope and the monitor 18. Hence this monitor can be placed in a central office whereby the hybrid stethoscope carried by the doctor can be at the bed of a patient remote from the central office.

Monitor 18 includes an LCD device 19, or a like device, which exhibits the same waveform shown in the visual display module, but in a larger scale along a millisecond scale, as shown in FIG. 4.

The data acquired by monitor 18 is valuable not only to facilitate a diagnosis of the disorder responsible for the internal body sounds but also for providing a record of the data and the diagnosis based thereon. To this end, data from monitor 18 is digitized and then downloaded into the data bank of a dedicated computer 21 having a keyboard 22 by means of which the physician can enter his diagnosis of the disorder and the prescribed treatment. Hence, stored in computer 21 is the waveform of body sounds emanating from a particular patient, an analysis of this waveform, and a diagnosis and treatment based on the analysis.

The data stored in computer 21 can be printed out in a printer 23, so that the data can be circulated to consultants and others having an interest in the patient's condition. And the data can be conveyed to the web sites of health care management organizations on an Internet highway.

The same monitor can service several hybrid stethoscopes, in which event it is necessary to identify each stethoscope. The data transmitted from each stethoscope can be preceded by an identifying bar code which will then appear in the data base next to the data derived from the stethoscope.

Modifications:

Module 14 shown in FIG. 5 is only capable of transmitting waveform data to external sites such as monitor 18 by means of a microwave transmitter whose signals are intercepted by a receiver in the monitor.

However in some instances, it is desirable for modules attached to different stethoscopes to be capable of communicating with each other. To this end, it is only necessary to include in each module a microwave receiver tuned to the frequency of the microwave transmitter which is turned off when the module is in a receiving mode to pick up data being transmitted from a remote module.

Hence what is seen on the LCD screen of the local module is the waveform of sounds being picked up by a remote stethoscope.

Such intercommunication among modules makes it possible for a physician at a remote site to see the waveform of sounds being picked up by a physician at a local site and thereby be in a position to assist the local physician in analyzing the waveforms.

Another useful modification is to include in the module a push-button controlled solid state unit adapted to freeze and store an existing waveform being exhibited. In this way, the examining physician can later analyze in depth the selected waveform. This freezing action is reserved for waveforms that appear to be of exceptional significance.

External Sites:

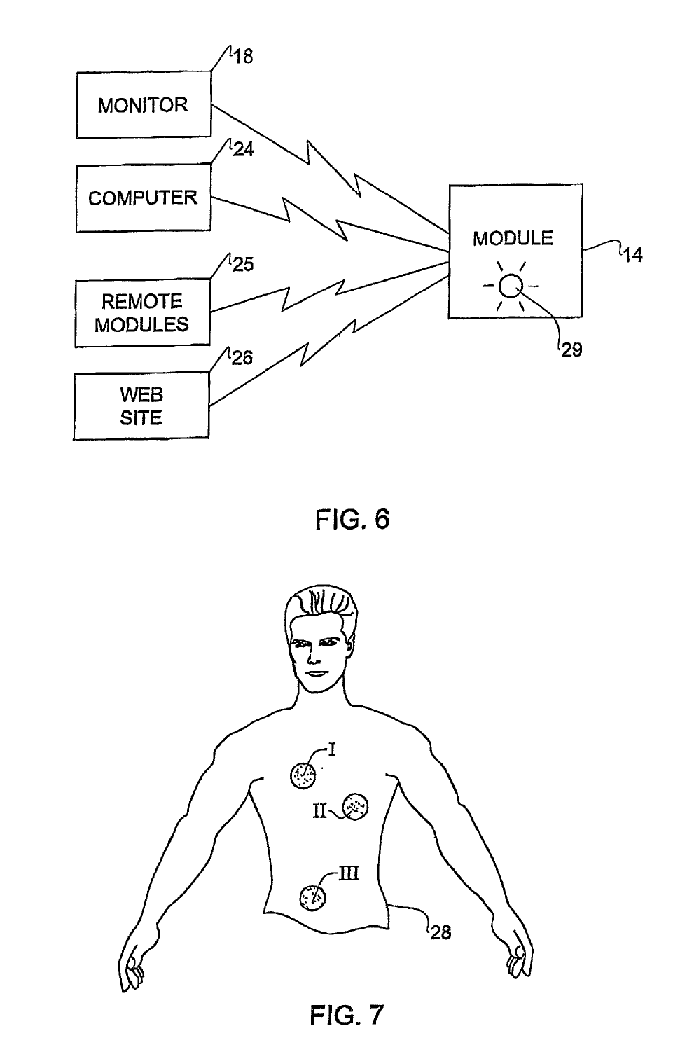

As indicated in FIG. 6, module 14 attached to the bell or stethoscope is provided with a microwave transmitter to convey the data to diverse external sites all of which are provided with a receiver tuned to the frequency of the module transmitter.

One of these sites is the external monitor 18, previously described. Another external site is the personal computer 24 of the examining physician in which is stored not only the waveform of the sounds emanating from the patient being examined but other medical data derived from the same patient. Thus when necessary, the physician can obtain from computer 24 a printout of a patient's medical record.

Module 14 can also communicate with remote modules 25 in the manner previously explained, in which case the modules, in addition to having a microwave transmitter must also be provided with a microwave receiver.

And module 14 may be linked by microwave transmissions to a website 26 on an Internet highway. The website may for example be a healthcare organization that should be informed of a stethoscopic examination conducted by a physician who is a member of this organization.

Respiratory Sounds:

Breathing sounds produced by the lungs during respiration are of diagnostic significance. Thus a patient who has difficulty breathing will usually produce wheezing or whistling sounds indicative of an abnormal condition. The diagnostic value of listening to chest sounds has been known to medicine since the time of Hippocrates in the fifth century BC.

The advantage that a modem physician has over Hippocrates is that he can by means of a stethoscope placed over the chest of a patient concentrate his attention on respiratory sounds. However, as pointed out previously, an acoustic stethoscope functions as a low-pass filter which attenuates the high-frequency components of the sounds heard by the user. Since wheezing and whistling sounds produced by abnormal respiratory conditions fall mainly in the high-frequency sonic range, they will not clearly be heard by the physician using an acoustic stethoscope.

However, bell 10 of the stethoscope does pick up the full frequency range of breathing sounds when the bell is placed over the chest of the patient, and what the bell 10 hears is picked up by microphone 16 in the module 14 attached to the bell. Hence the ausculation signal S yielded by microphone 16 is faithful to the respiratory sounds.

Signal S is transmitted from module 14 to the remote monitor 18 on whose LCD screen will be displayed the waveform of the ausculation signal. The received signal is also fed to computer 21 where it is recorded by a solid state or tape recorder 27. This recording of breathing sounds can later be heard by a physician then diagnosing the patient's condition

Multi-Mode Operation

Ausculation by means of a stethoscope is not limited to the diagnosis of cardiac, vascular or lung disorders, for it is useful in diagnosing any abnormal condition giving rise to internal sounds loud enough to be picked up by an acoustic stethoscope. In order therefore to operate a hybrid stethoscope in accordance with the invention in any one of several diagnostic modes, each of which is reserved for a particular region of the body, module 14 as shown in FIG. 6, is provided with a knob-operated mode selector switch 28.

Switch 28 is operatively coupled to a code generator (not shown) which acts to preface the ausculation signal from the microphone which is being transmitted with a binary code signal which identifies the mode of the ausculation signal.

Thus by way of example, FIG. 7 shows the body 28 of a patient who is to be examined in three modes. Mode I is the Chest Mode which takes effect when the bell of the stethoscope is placed over the chest of the patient to pick up the sounds of breathing. Mode II is the Cardiac Mode which is in effect when the bell of the stethoscope is placed over the heat of the patient to hear sounds emanating from the region of the heart. Mode III is the Abdominal Mode in which the bell is placed over the belly of the patient to pick up gurgling and other sounds accompanying abdominal activity. There can of course be additional modes.

By coding the mode, each ausculation signal is identified, thereby facilitating its later retrieval. Thus if a doctor some weeks after a Mode I examination wishes to hear the sounds of breathing recorded at the time of the examination, he has only to consult the patient's file as presented on the computer screen, and select from this file the desired mode recording.

This multi-mode arrangement makes it possible to conduct a full stethoscopic examination of a patient, to record the sounds emanating from sounds produced in various internal regions of a given patient and to later retrieve any one of these recordings, as well as the visual waveforms of the recorded sounds.

While there has been shown preferred embodiments of the invention, it is to be understood that many changes may be made therein without departing from the essential spirit of the invention. Thus some of the analytical functions carried out in the external monitor such as a reading of pulse rate can be carried out by means of an integrated-circuit chip included in the module, in which case data produced by the chip call be presented on the LCD screen of the module above or below the waveform or the data may be recorded in a solid state memory in the chip and later downloaded into the data bank of the computer.

Instead of mounting the LCD module on the bell of the stethoscope to display the waveform of the cardiac or other internal sounds picked up by the bell, the LDC module can be an independent unit which is wired to the stethoscope. In this way a physician can with one hand apply the stethoscope to the body of a patient and listen to the sounds picked up by the stethoscope, while holding the LCD module in his other hand to observe the waveform display. And instead of a wired connection between the LCD display and the stethoscope, a wireless fared or microwave interconnection may be used to effect this coupling.

1. A hybrid stethoscope comprising:
A. a bell placeable by a user on a body site overlying an internal region to receive sounds emanating therefrom;
B. a tubular line acoustically coupling the bell to earphones insertable in ears of the user to cause the user to hear said sounds; and
C. a visual display module mounted on the bell and responsive to sounds received thereby and adapted to exhibit an analog waveform of the sounds whereby the user effectively sees as well as hears the sounds.

2. The stethoscope as set forth in claim 1 in which the module is a battery-powered self-sufficient unit.

3. The stethoscope as set forth in claim 1 in which the module is mounted on a rear surface of the bell.

4. The stethoscope as set forth in claim 2 in which said module includes a liquid crystal display device to exhibit said waveform.

5. The stethoscope as set forth in claim 4 in which the module includes a microphone which intercepts sounds received by the bell to produce an audio signal that is applied to the liquid crystal display device.

6. The stethoscope as set forth in claim 5 in which the tubular line functions as a low-pass filter to filter out high-frequency components of the sounds.

7. The stethoscope as set forth in claim 6 in which the microphone of the module is sensitive to said high-frequency components which are included in said audio signal.

8. The stethoscope as set forth in claim 1 in which the bell is double headed, opposing heads of the bells being joined at a junction to which said module is laterally attached.

9. The stethoscope as set forth in claim 1 in which said module includes a transmitter to convey said audio signal to an external monitor adapted to exhibit said waveform and to manipulate said waveform to effect measurements which aid in an analysis thereof.

10. The stethoscope as set forth in claim 9 in which said transmitter is a microwave transmitter and said monitor is provided with a microwave receiver to receive and demodulate the transmission to derive the audio signal therefrom.

11. An intercommunication system comprising a plurality of hybrid stethoscopes as set forth in claim 1 provided with means intercoupling the visual display modules whereby the waveform produced by any module in the system is exhibited in other modules thereof.

12. A system as set forth in claim 11 in which each module includes a microwave transmitter to transmit the waveform to the other modules, and a receiver to receive the transmitted waveform.

13. A system for conducting a stethoscopic examination using a hybrid stethoscope as set forth in claim 5 in which the audio signal yielded by the microphone in the module is transmitted to a remote station at which it is recorded.

14. A system as set forth in claim 13 in which the body site at which the bell is placed is the chest of the patient, and the resultant audio signals are breathing sounds.

15. A system as set forth in claim 13 in which the system is selectively operable in several modes, each of which depends on the body site on which the bell is placed said module including means to preface the signal yielded by the microphone in a selected mode with a code signal identifying the mode.
